(19) United States
(12) Patent Application Publication
(10) Pub. No.: US 2011/0060056 A1
(43) Pub. Date: Mar. 10, 2011

(54) Inhibition of the Survival of Gastric Cancer by Cyclohexenone Compounds from Antrodia Camphorata

(75) Inventors: Sheng-Yun Liu, Taipei Hsien (TW); Wu-Che Wen, Taipei Hsien (TW); Mao-Tien Kuo, Taipei Hsien (TW)

(73) Assignee: GOLDEN BIOTECHNOLOGY CORPORATION, Taipei Hsien (TW)

(21) Appl. No.: 12/875,545

(22) Filed: Sep. 3, 2010

(30) Foreign Application Priority Data
Sep. 9, 2009 (TW) 098130425

(51) Int. Cl. A61K 31/122; A61P 35/00; C12N 5/00

(52) U.S. Cl. 514/690; 435/375

(57) ABSTRACT

The present invention relates to a novel application of a compound. The compound 4-hydroxy-2,3-dimethoxy-6-methyl-5-(3,7,11-trimethyl-dodeca-2,6,10-trienyl)-cyclohex-2-enone of the invention is isolated and purified from the extracts of Antrodia camphorata, which can be applied for inhibiting the survival of gastric cancer cells and be used as a pharmaceutical composition to inhibit the gastric tumor growth.

### BACKGROUND OF THE INVENTION

1. Field of the Invention

The present invention relates to a new application for inhibiting cancer cell survival, in particular to the application for inhibiting the survival of gastric cancer cells by a compound isolated and purified from Antrodia camphorata.

2. The Prior Arts

Gastric cancer is the most common type of malignant neoplasm in digest system. According to statistical analysis, gastric cancer is the fifth leading cause of cancer death in Taiwan and mostly occurs in the ages over 45 years old. In addition, the incidence of gastric cancer in male is higher than that in female. The risk factors for developing gastric cancer include genetic factors, individual disease history such as aplastic anemia or chronic atrophic gastritis, wrong eating habits such as scanty dietary intake of fresh fruits and vegetables or taking pickled food for a long period of time, infection of Helicobacter pylori, smoking and alcohol abuse. Over 95 percent of gastric cancer belongs to adenocarcinoma formed by epithelial cells of gastric mucosa; therefore the so called gastric cancer means adenocarcinoma. According to the invasion level of cancer cells, gastric cancer can also be classified as early gastric cancer which the cancer cells are restricted to mucosa or submucosa and advanced gastric cancer which the cancer cells already invade to inherent myometrium or entry to deeper tissues.

The clinical syndromes of patients with early gastric cancer are not specific and the uncomfortable physical conditions caused by gastric cancer are not significantly different from that caused by general peptic ulcer. Therefore, most cases will not be defined as gastric cancer until the terminal cancer stage. At the terminal cancer stage, the major syndromes include weight loss, abdominal pain, nausea, loss of appetite, dysphagia, black excrement, easy to feel satiated, gastric ulcer-like pain or leg edema. The current clinical therapy for gastric cancer includes gastrectomy, chemotherapy and radiotherapy, wherein the excision of tumor as well as its surrounding tissues and lymph nodes by surgery is the only way to totally cure gastric cancer patients. However, except the survival rate of early staged gastric cancer patients can reach 50 percent after surgical excision, the survival rate of most of the gastric cancer patients diagnosed with advanced gastric cancer is far less than 50 percent after surgical excision. As to invasive and recurrent gastric cancer which cannot be treated by surgical excision, the supportive therapy of chemotherapy and radiotherapy will be the only recourse to partially control the cancer. However, the prognosis is quite poor and the 5-year survival rate is even lower than 15 percent. Moreover, the recurrence and metastasis of gastric cancer, side effects resulted from chemical drugs, resistance to chemical drugs or uncomfortable symptoms induced by radiotherapy are also the common clinical thorny problems.

Due to the high incidence of distant metastases such as transperitoneal extension and liver metastasis, the prognosis of patients is still not optimistic even treated intensively by surgery or other methods. Thus gastric cancer still belongs to one kind of cancers with high mortality though its global incidence has been decreasing gradually in the last ten years. Gastric cancer indeed becomes a health-threatening and important issue for the people in the whole world. Therefore, it is urgently needed to find a nature therapeutic substance that doesn't cause side effects, and can enhance the therapeutic efficacy and avoid the derivative disorders.

Antrodia camphorata is also known as various names such as Chang-Chih, Ganoderma comphoratum, Antrodia camphorata, Taiwanofungus camphorata, and Camphor Mushroom . . . etc., a genus of Basidiomycoya, Homobasidiomycetes, Aphyllophorales, Polyporaceae, and Antrodia in Fungi, and also a perennial mushroom. It is a Taiwan endemic species of fungi and received its name because it only grows on the inner wall of the hollow material from Taiwan's endemic Lauraceae tree species, Cinnamomum kanehirai. The price of Antrodia camphorata is very high due to the extremely slow growth rate of natural Antrodia camphorata.

The fruiting bodies of Antrodia camphorata are perennial, sessile, hard and woody, which exhale strong smell of sassafras (camphor aroma). The appearances are various with plate-like, bell-like, hoof-like, or tower-like shapes. They are reddish in color and flat when young, attached to the surface of wood. Then the brims of the front end become reversely curled tilting and extending to the surroundings. At the same time, the color turns to be faded red-brown or cream yellow brown, with ostioles all over. This region is of very high medical value.

In traditional Taiwanese medicine, the curative effects of Antrodia camphorata include removing rheumatism, smoothing vitality, nourishing blood, eliminating bruises, benefiting spleen and stomach, lessening accumulation, detoxification, subsiding swelling, sedation and relieving pain, and is used as a great antidote for detoxifying food poisoning, diarrhea, vomiting and pesticide poisoning. Furthermore, it has adjuvant therapeutic effects on liver and stomach dysfunction and the diseases of blood circulation. Antrodia camphorata, like general edible and medicinal mushrooms, is rich in numerous nutrients including polysaccharides (such as β-glucosan), triterpenoids, superoxide dismutase (SOD), adenosine, proteins (immunoglobulins), vitamins (such as vitamin B, nicotinic acid), trace elements (such as calcium, phosphorus and germanium and so on), nucleic acid, agglutinin, amino acids, steroids, lignins and stabilizers for blood pressure (such as antrodia acid) and so on. These physiologically active ingredients are believed to exhibit effects such as: anti-tumor activities, increasing immuno-modulating activities, anti-allergy, anti-bacteria, anti-hypertension, decreasing blood sugar, decreasing cholesterol, etc.

Triterpenoids are the most studied components among the numerous compositions of Antrodia camphorata. Triterpenoids are the summary terms for natural compounds, which contain 30 carbon atoms with the pent- or hex-acyclic structures. The bitter taste of Antrodia camphorata is from the component of triterpenoids. Three novel ergostane-type triterpenoids (antcin A, antcin B, antcin C) were isolated by Cherng et al. from the fruiting bodies of Antrodia camphorata (Cherng, I. H., and Chiang, H. C. 1995. Three new triterpenoids from Antrodia cinnamomea. J. Nat. Prod. 58:365-371). Three new compounds zhankuic acid A, zhankuic acid B and zhankuic acid were extracted from the fruiting bodies of Antrodia camphorata with ethanol by Chen et al. (Chen, C. H., and Yang, S. W. 1995. New steroid acids from Antrodia cinnamomea,—a fungus parasitic on Cinnamomum micranthum. J. Nat. Prod. 58:1655-1661). In addition, Cherng et al. also found three other new triterpenoids from the fruiting bodies of Antrodia camphorata, which are sesquiterpene lactone and 2 biphenyl derived compounds, 4,7-dimethoxy-5-methyl-1,3-benzodioxole and 2,2′,5,5′-teramethoxy-3,4,3′,4′-bi-methylenedioxy-6,6′-dimethylbiphenyl (Chiang, H. C., Wu, D. P., Cherng, I. W., and Ueng, C. H. 1995. A sesquiterpene lactone, phenyl and biphenyl compounds from Antrodia cinnamomea. Phytochemistry. 39:613-616). In 1996, four novel ergostane-type triterpenoids (antcins E and F and methyl antcinates G and H) were isolated by Cherng et al. with the same analytic methods (Cherng, I. H., Wu, D. P., and Chiang, H. C. 1996. Triteroenoids from Antrodia cinnamomea. Phytochemistry. 41:263-267). And two ergostane related steroids, zhankuic acids D and E together with three lanosta related triterpenes, 15 alpha-acetyl-dehydrosulphurenic acid, dehydroeburicoic acid, and dehydrosulphurenic acid were isolated by Yang et al. (Yang, S. W., Shen, Y. C., and Chen, C. H. 1996. Steroids and triterpenoids of Antrodia cinnamomea—a fungus parasitic on Cinnamomum micranthum. Phytochemistry. 41:1389-1392).

Although Antrodia camphorata extracts were reported to have the above mentioned effects from the previously published experimental results, and the several compounds were analyzed and identified successfully, further works are needed to identify the effective compounds to inhibit cancer growth and thus to contribute beneficial effects on cancer therapy such as the treatment and prevention of gastric cancer.

### SUMMARY OF THE INVENTION

In order to identify the anti-cancer compounds from the extracts of Antrodia camphorata, the compound of the formula (1) was isolated and purified in the present invention,

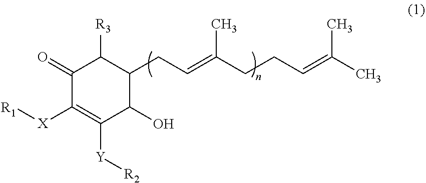

wherein X and Y can be oxygen, nitrogen or sulfur, R₁, R₂ and R₃ are each a hydrogen atom, methyl or (CH₂)ₘ—CH₃ and m=1-12; n=1-12.

A preferred compound of the general formula (1) is 4-hydroxy-2,3-dimethoxy-6-methyl-5-(3,7,11-trimethyl-dodeca-2,6,10-trienyl)-cyclohex-2-enone as shown in formula (2), with molecular formula of C₂₄H₃₈O₄, appearance of pale yellow powder and molecular weight of 390.

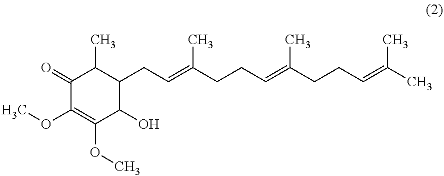

Cyclohexenone compounds having the structures of formula (1) and formula (2) are purified from aqueous extraction or organic solvent extraction of Antrodia camphorata. The organic solvents used include, but not limited to, alcohols such as methanol, ethanol or propanol, esters such as ethyl acetate, alkanes such as hexane, or halogenated alkanes such as chloromethane, chloroethane. Among them, alcohol is preferred, and ethanol is particularly preferred.

Cyclohexenone compounds of the present invention are applied in inhibiting the survival of cancer cells, which can further be used as a pharmaceutical composition for treating cancer and to enhance the cancer therapeutic effects. The compounds of the invention can be applied in inhibiting the survival of gastric cancer cells, which result in delaying the growth of the cancer cells and suppressing proliferation of the cancer cells, and further inhibiting cancer deterioration. The preferred compound is 4-hydroxy-2,3-dimethoxy-6-methyl-5-(3,7,11-trimethyl-dodeca-2,6,10-trienyl)-cyclohex-2-enone of the formula (2).

On the other hand, the compounds of formula (1) and/or formula (2) in the present invention can be incorporated into pharmaceutical compositions or medicaments for treating gastric cancer to inhibit the survival of cancer cells. The pharmaceutical compositions include not only the compounds of formula (1) and/or formula (2), but also the pharmaceutically accepted carriers. Examples of such carriers include, but are not limited to, excipients such as water, fillers such as sucrose or starch, binders such as cellulose derivatives, diluents, disintegrants, absorption enhancers or sweeteners. The pharmaceutical composition or medicament can be manufactured through mixing the compounds of formula (1) and/or formula (2) with at least one of the carriers by means of conventional methods known in the pharmaceutically technical field, which can be formulated in the form of, but are not limited to, powder, tablets, capsules, pellets, granules or other liquid formulation.

The present invention is further explained in the following embodiment illustration and examples. Those examples below should not, however, be considered to limit the scope of the invention, it is contemplated that modifications will readily occur to those skilled in the art, which modifications will be within the spirit of the invention and the scope of the appended claims.

### DETAILED DESCRIPTION OF THE PREFERRED EMBODIMENT

The aqueous or organic solvent extracts of Antrodia camphorata were subjected to high-performance liquid chromatography (HPLC) for isolation and purification. Each fraction was recovered and applied to anti-cancer assay. The potent fractions with anti-cancer effects were analyzed for the composition and further assayed against gastric cancer cells. The above approach then led to the identification of compounds of formula (1) and formula (2) in inhibiting the survival of gastric cancer cells.

The compound 4-hydroxy-2,3-dimethoxy-6-methyl-5-(3,7,11-trimethyl-dodeca-2,6,10-trienyl)-cyclohex-2-enone of the formula (2) is explained below as an example for the present invention. The anti-cancer effects of 4-hydroxy-2,3-dimethoxy-6-methyl-5-(3,7,11-trimethyl-dodeca-2,6,10-trienyl)-cyclohex-2-enone was assessed using 3-(4,5-dimethylthiazol-2-yl)-2,5-diphenyltetrazolium bromide (MTT) assay according to the anti-cancer drug screening model of National Cancer Institute (NCI) to analyze survival rates on gastric cancer cell line TSGH-9201. These assays have proved that cyclohexenone compounds from Antrodia camphorata decreased the survival rates of gastric cancer cell line TSGH-9201, and simultaneously showed low half inhibition concentration (IC₅₀) value. Therefore, cyclohexenone compounds from Antrodia camphorata can be used for inhibiting the survival of gastric cancer cells and further be applied for the treatment of gastric cancer. The details of the examples are described as follows:

### Example 1

Isolation of 4-hydroxy-2,3-dimethoxy-6-methyl-5-(3,7,11-trimethyl-dodeca-2,6,10-trienyl)-cyclohex-2-enone

One hundred grams of mycelia, fruiting bodies or mixture of both from Antrodia camphorata were placed into a flask. A proper amount of water and alcohol (70-100% ethanol solution) was added into the flask and were stirred at 20-25° C. for at least 1 hour. The solution was filtered through both a filter paper and a 0.45 μm membrane, and then collected as the extract.

The extract of Antrodia camphorata was subjected to High Performance Liquid chromatography (HPLC) analysis. The separation was performed on a RP18 column using a mobile phase consisted of methanol (A) and 0.1-0.5% acetic acid (B), with the gradient conditions: the ratio of (B) from 95% to 20% 0-10 minutes, from 20% to 10% 10-20 minutes, kept 10% 20-35 minutes, and increased from 10% to 95% 35-40 minutes at the flow rate of 1 ml/min. The column effluent was monitored with a UV-visible detector.

The fractions collected during 25-30 min were concentrated to yield 4-hydroxy-2,3-dimethoxy-6-methyl-5-(3,7,11-trimethyl-dodeca-2,6,10-trienyl)-cyclohex-2-enone, a product of pale yellow powder. The analysis of 4-hydroxy-2,3-dimethoxy-6-methyl-5-(3,7,11-trimethyl-dodeca-2,6,10-trienyl)-cyclohex-2-enone showed the molecular formula of C₂₄H₃₈O₄, molecular weight of 390, and melting point of 48° C.˜52° C. Investigation of NMR spectra showed that ¹H-NMR (CDCl₃) δ(ppm)=1.51, 1.67, 1.71, 1.75, 1.94, 2.03, 2.07, 2.22, 2.25, 3.68, 4.05, 5.07, and 5.14; ¹³C-NMR (CDCl₃) δ(ppm)=12.31, 16.1, 16.12, 17.67, 25.67, 26.44, 26.74, 27.00, 39.71, 39.81, 4.027, 43.34, 59.22, 60.59, 120.97, 123.84, 124.30, 131.32, 135.35, 135.92, 138.05, 160.45, and 197.12.

### Example 2

### In Vitro Survival Assay for Anti-Gastric Cancer Effects

Inhibiting effects of gastric cancer cells by cyclohexenone compounds of Antrodia camphorata from example 1 were assessed according to the anticancer-drug screening model of National Cancer Institute (NCI). The compound 4-hydroxy-2,3-dimethoxy-6-methyl-5-(3,7,11-trimethyl-dodeca-2,6,10-trienyl)-cyclohex-2-enone from example 1 was added into the culture media of gastric cancer cell line TSGH-9201 to determine the survival rates. Survival of cell was analyzed using MTT assay. TSGH-9201 cell line was a human gastric carcinoma cell line.

MTT assay is commonly used to analyze cell proliferation, survival rate of viable cells and cytotoxicity. MTT (3[4,5-dimethylthiazol-2-yl]-2,5-diphenyltetrazolium bromide) is a yellow dye which can be converted to water-insoluble purple formazan on the reductive cleavage of its tetrazolium ring by the succinate tetrazolium reductase in mitochondria of cells. The amount of formazan produced is used to detect the number of viable cells and calculate the survival rates.

The TSGH-9201 cells were cultivated in DMEM medium supplemented with 10% fetal bovine serum, 100 U/ml of Penicillin and 100 μg/ml of Streptomycin at 37° C., 5% CO₂ for 24 hours. Proliferated cells were washed once with PBS, treated with 1× trypsin-EDTA, and centrifuged at 1200 rpm for 5 min. The supernatant was removed and the cell pellet was resuspended in 10 ml of fresh medium by gently shaking. Cells were seeded onto 96-well plates. Cells treated with the crude extracts of Antrodia camphorata (total ethanol extracts, not purified) were designed as the control group; and cells treated with 4-hydroxy-2,3-dimethoxy-6-methyl-5-(3,7,11-trimethyl-dodeca-2,6,10-trienyl)-cyclohex-2-enone were designed as the experiment group. Both substrates were added in the concentration of 30, 10, 3, 1, 0.3, 0.1 and 0.03 μg/ml respectively. Cells were cultivated at 37° C., 5% CO₂ for 48 hours. Afterward, 2.5 mg/ml of MTT solution was added to each well and incubated in the dark for 4 hours, followed by the addition of 100 μl of lysis buffer to stop the reaction. The absorbances were measured at 570 nm with an ELISA Reader to determine the survival rates. The half inhibition concentration (IC₅₀) value was also calculated and listed in Table 1.

TABLE 1
Results of in vitro survival assay for inhibition of gastric cancer cells

| Sample | IC₅₀ (μg/ml) |
|---|---|
| Experiment group (formula 2) | 1.32 |
| TSGH-9201 | |

Refers to the result of table 1, the IC₅₀ value of 4-hydroxy-2,3-dimethoxy-6-methyl-5-(3,7,11-trimethyl-dodeca-2,6,10-trienyl)-cyclohex-2-enone toward TSGH-9201 was 1.32 μg/ml, which was significantly lower than those of total extracts from Antrodia camphorata (data not shown). Therefore, 4-hydroxy-2,3-dimethoxy-6-methyl-5-(3,7,11-trimethyl-dodeca-2,6,10-trienyl)-cyclohex-2-enone from Antrodia camphorata can be utilized to inhibit the survival of gastric cancer cells.

In summary, the compound 4-hydroxy-2,3-dimethoxy-6-methyl-5(3,7,11-trimethyl-dodeca-2,6,10-trienyl)-cyclohex-2-enone isolated from Antrodia camphorata according to the present invention can be used to effectively inhibit the survival of human gastric cancer cells. The cyclohexenone compounds from Antrodia camphorata won't induce uncomfortable side effects, toxicity or complications when being applied for treating gastric cancer. Moreover, these compounds of the invention can also be used concurrently with chemotherapy drugs when treating gastric cancer in order to reduce the using amount of chemotherapy drugs as well as decreasing the side effects resulted from chemotherapy drugs. In addition, it can be incorporated into pharmaceutical compositions. The pharmaceutical compositions include not only effective amount (or active dose) of the cyclohexenone compounds from Antrodia camphorata of the present invention, but also the pharmaceutically accepted carriers. Examples of such carriers include, but are not limited to, excipients such as water, fillers such as sucrose or starch, binders such as cellulose derivatives, diluents, disintegrants, absorption enhancers or sweeteners. The composition of the present invention can be manufactured through mixing the compound of cyclohexenone from Antrodia camphorata with at least one of the carriers by means of conventional methods known in the pharmaceutically technical field, and can be formulated in the forms of powder, tablets, capsules, pellets, granules or other liquid formulation, but are not limited to. The purpose for treating gastric cancer can then be accomplished.

What is claimed is:

1. A method of inhibiting the survival of gastric cancer cells, comprising administering to a subject in need thereof an effective amount of a compound having the following formula: 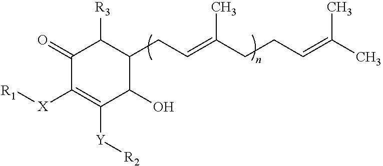
wherein X and Y is oxygen, nitrogen or sulfur, R₁, R₂ and R₃ are each a hydrogen atom, methyl or (CH₂)ₘ—CH₃, and m=1-12; n=1-12.

2. The method as claimed in claim 1, wherein the compound is 4-hydroxy-2,3-dimethoxy-6-methyl-5-(3,7,11-trimethyl-dodeca-2,6,10-trienyl)-cyclohex-2-enone.

3. The method as claimed in claim 2, wherein the compound is isolated from Antrodia camphorata.

4. The method as claimed in claim 3, wherein the compound is isolated from the aqueous extracts of Antrodia camphorata.

5. The method as claimed in claim 3, wherein the compound is isolated from the organic solvent extracts of Antrodia camphorata.

6. The method as claimed in claim 5, wherein the organic solvents are selected from the group consisting of alcohols, esters, alkanes, and halogenated alkanes.

7. The method as claimed in claim 6, wherein the alcohol is ethanol.

8. The method as claimed in claim 1, wherein the gastric cancer cells are from TSGH-9201 cell line.

9. The method as claimed in claim 1, wherein the compound is administered in a form selected from the group consisting of powder, tablet, capsule, pellet, granule and liquor.

10. A pharmaceutical composition for inhibiting the survival of gastric cancer cells comprising an active dose of compound as claimed in claim 1 and a pharmaceutically acceptable carrier.

11. The composition as claimed in claim 10, wherein the compound is 4-hydroxy-2,3-dimethoxy-6-methyl-5-(3,7,11-trimethyl-dodeca-2,6,10-trienyl)-cyclohex-2-enone.

12. The composition as claimed in claim 11, wherein the compound is isolated from Antrodia camphorata.

13. The composition as claimed in claim 12, wherein the compound is isolated from the aqueous extracts of Antrodia camphorata.

14. The composition as claimed in claim 12, wherein the compound is isolated from the organic solvent extracts of Antrodia camphorata.

15. The composition as claimed in claim 14, wherein the organic solvents are selected from the group consisting of alcohols, esters, alkanes, and halogenated alkanes.

16. The composition as claimed in claim 15, wherein the alcohol is ethanol.

17. The composition as claimed in claim 10, wherein the gastric cancer cells are from TSGH-9201 cell line.

18. The composition as claimed in claim 10, wherein the composition is in a form selected from the group consisting of powder, tablet, capsule, pellet, granule and liquor.
